(19) United States
(12) Patent Application Publication
(10) Pub. No.: US 2019/0021989 A1
(43) Pub. Date: Jan. 24, 2019

(54) GALENIC FORMULATION COMPRISING A TOPICAL DRUG

(71) Applicants: Catherine CANTINA, Blotzheim (FR); Paul FERNANDES, Sierentz (FR); Melinda Eniko GRUBESA, Divonne les Bains (FR); Claire HAUG, Saint-Louis (FR); Michael KELLER, Basel (CH); Isabelle RAULT, Antibes (FR)

(72) Inventors: Catherine CANTINA, Blotzheim (FR); Paul FERNANDES, Sierentz (FR); Melinda Eniko GRUBESA, Divonne les Bains (FR); Claire HAUG, Saint-Louis (FR); Michael KELLER, Basel (CH); Isabelle RAULT, Antibes (FR)

(73) Assignee: NOVARTIS AG, Basel (CH)

(21) Appl. No.: 16/070,568

(22) PCT Filed: Jan. 21, 2016
(86) PCT No.: PCT/IB2016/050293

§ 371 Date: Jul. 17, 2018

(30) Foreign Application Priority Data
Jan. 21, 2015 (EP) 15151973.3

(51) Int. Cl. A61K 9/00; A61K 9/06; A61K 31/4745; A61K 47/12; A61K 47/10; A61K 47/02

(52) U.S. Cl. CPC A61K 9/0014; A61K 9/06; A61K 47/02; A61K 47/12; A61K 47/10; A61K 31/4745

(57) ABSTRACT

The present invention relates to pharmaceutical compositions comprising a drug for topical administration, e.g. a TLR7 modulator. More specifically it relates to a pharmaceutical composition comprising a benzo[f][1,7]naphthyridine derivative.

The present invention relates to pharmaceutical compositions comprising a drug for topical administration, e.g. a TLR7 modulator. More specifically it relates to a pharmaceutical composition comprising a benzo[f][1,7]naphthyridine derivative.

### BACKGROUND OF THE INVENTION

For potent pharmaceutically effective compounds which are designed for topical administration, oftentimes low local tolerability is being observed. It is therefore of vital importance of providing a topical formulation that solves local intolerability and does not negatively impact efficacy of a corresponding compound.

### SUMMARY OF THE INVENTION

The galenic formulations of the present invention are pertaining to highly potent pharmaceutically effective compounds, such as a TLR7 modulator, which are adapted to topical administration, e.g. as a cream, gel or the like, which are stable and which have good skin tolerability.

The present invention provides in particular a pharmaceutical composition suitable for topical administration, comprising a pharmaceutically effective drug, e.g. a TLR7 modulator, e.g. a benzo[f][1,7]naphthyridine derivative e.g. a compound according to any one of formulae (I, (II), (III) and/or (IV), a solvent, an emulsifier, a thickener or gelling agent, a buffer, an alkalizing agent, a preservative and optionally an antioxidant.

### DETAILED DESCRIPTION OF THE INVENTION

The pharmaceutical compositions of the present invention are typically stable and generally well tolerated on skin.

As used herein, a TLR7 modulator may have the chemical structure as depicted below, i.e. a compound of formula (I):

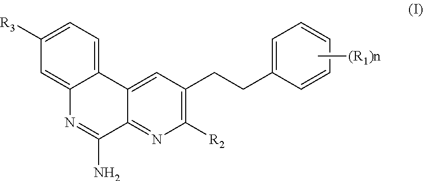

wherein each R₁, R₂, and R₃ are independently selected from H, —CH₃, —CH₂CH₃, —CF₃, —CH₂OH—OCH₃, —COOCH₃, —COOCH₂CH₃, F, Cl, Br, —CH₂OCH₃, —CH₂OCH₂CH₃, —N(CH₃)₂, —((O(CH₂)₂)₂—OH, —O(CH₂)₂—OH, —O(CH₂)₂—(PO₃H₂), —O(CH₂)₂—COOH, —O(CH₂)₂—CH(CH₃)₂, C₂-C₆-alkyl substituted with 1-3 substituents selected from —OH, —CH₃, cyclo-propyl, —O(CH₂)₂—COOH, —O(CH₂)₂—(PO₃H₂), —COOH, —COOCH₃, and —COOCH₂CH₃; and n is 0, 1,2 or 3; or a pharmaceutically acceptable salt thereof.

More specifically, a TLR7 modulator may have the chemical structure (II), (III), and/or (IV) as shown below or a pharmaceutically acceptable salt thereof.

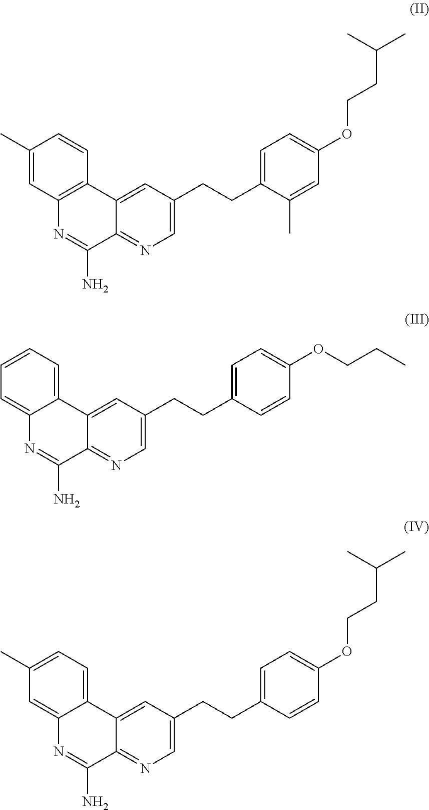

Examples of pharmaceutically acceptable salts of the compounds of formula I-IV include salts with inorganic acids, such as hydrochloride, hydrobromide and sulfate, salts with organic acids, such as acetate, fumarate, maleate, benzoate, citrate, malate, methanesulfonate and benzenesulfonate salts, or, when appropriate, salts with metals, such as sodium, potassium, calcium and aluminium, salts with amines, such as triethylamine and salts with dibasic amino acids, such as lysine. A particular salt may be a hydrochloride.

A pharmaceutical composition of the present invention typically contains from 0.001 to 5% by weight of a TLR7 modulator, or from 0.001 to 1% by weight, or from 0.01 to 0.5% by weight, or e.g. from 0.003 to 0.1% by weight, based on the total weight of the composition.

A composition of the present invention typically comprises one or more excipients, such as a solvent, an emulsifier, a thickener or gelling agent, a buffer, an alkalizing agent, a preservative and/or an antioxidant.

### Definitions

As used herein a solvent may be typically selected from water, ethanol, iso-propanol, n-propanol, propylene glycol, ethylene glycol, diethylene glycol, diethylene glycol monoethyl ether (Transcutol® HP), diethylene glycol diethyl ether, corn oil, or benzyl alcohol. In an embodiment water, ethanol, propylene glycol, diethylene glycol monoethyl ether and/or benzyl alcohol may be used individually or in any combination thereof.

A solvent is typically present in an amount of about 0.1-95% weight, or from 1-95% weight, or from 5-95% by weight, or from 15-95% by weight, or from 25-95% by weight, or from 35-95% by weight, or from 45-95% by weight, or from 55-95% by weight, based on the total weight of the composition.

As used herein, an emulsifier may typically prevent the separation of the ingredients within a pharmaceutical composition and may also extend shelf life (time of storage). An example of such an emulsifier is lecithin, also called phosphatidyl choline, phosphatidyl ethanolamine, cholesterol, cetylalcohol, polyoxyl stearyl ether (e.g. Brij® S2, Brij® S721), or caprylcaproyl polyoxyl glyceride (Labrasol®). In an embodiment lecithin, polyoxyl stearyl ether, caprylcaproyl polyoxyl glyceride and/or cholesterol may be used individually or in any combination thereof.

An emulsifier is typically present in an amount of about 0.05-15% weight, or from 0.1-10% weight, or from 0.1-8% by weight, based on the total weight of the composition.

As used herein, a thickener or aelling agent may serve multiple purposes such as thickening, gelling, emulsifying and/or stabilizing and may for example be selected from a cellulose such as carboxymethyl cellulose, or hydroxyethyl cellulose; polyacrylates e.g. carbomer or carbopol (e.g. Carbopol® 974); polycarbophils e.g. Noveon® AA-1; polyvinylalcohol such as Mowiol® 26-88; polyvinylpyrrolidone such as Povidone® K30; an acrylamide sodium/acryloyldimethyl taurate copolymer such as Sepineo® P600; and xanthan gum. In an embodiment Sepineo® P600 and xanthan gum may be used individually or together.

Such a thickener or gelling agent is typically present in an amount of about 0.05-10% weight, or from 0.1-7% weight, or from 0.1-5% by weight, based on the total weight of the composition.

As used herein, a buffer may typically serve the adjustment of the pH over time, preferably to a physiological pH. Examples of buffer substances are acetate, ascorbate, borate, hydrogen carbonate/carbonate, citrate, gluconate, lactate, phosphate, propionate and TRIS (tromethamine) buffers. In an embodiment, citric acid buffer is being used. The amount of buffer substance added is, typically, the amount necessary to ensure and maintain a physiologically tolerable pH range. The physiologically tolerable pH range is generally in the range of from 4 to 9, or from 4.5 to 8.5, or from 5.0 to 8.2.

As used herein, an alkalizing agent may typically serve the adjustment of the pH, preferably to a physiological pH. Examples of alkalizing agents are sodium hydroxide, ammonium hydroxide, triethylamine, or tris(2-hydroxyethyl)amin. The amount of alkalizing agent added is, typically, the amount necessary to ensure a physiologically tolerable pH range. The physiologically tolerable pH range is generally in the range of from 4 to 9, or from 4.5 to 8.5, or from 5.0 to 8.2.

As used herein, a preservative, if desired, may for instance be a quaternary ammonium compound such as benzalkonium chloride (N-benzyl-N—(C₈-C₁₈alkyl)-N,N-dimethylammonium chloride), benzoxonium chloride or preservatives different from quaternary ammonium salts like parabens, such as, for example, methylparaben or propylparaben, alcohols, such as, for example, chlorobutanol, benzyl alcohol, phenoxy ethanol or phenyl ethanol, guanidine derivatives, such as, for example, chlorohexidine or polyhexamethylene biguanide, sodium perborate, Germal®II or sorbic acid. In an embodiment phenoxyethanol may be used.

The amount of preservative present is typically addressing an amount present to render a composition effectively preserved. A composition typically contains, for instance from 0.01 to 10% by weight, or from 0.1 to 10% by weight, or from 0.1 to 5% by weight, or from 0.1 to 2% by weight, based on the total weight of the composition.

As used herein, an antioxidant may be selected for example from ascorbic acid, acetylcysteine, cysteine, sodium hydrogen sulfite, butyl-hydroxyanisole, butyl-hydroxytoluene or alpha-tocopherol acetate.

In an embodiment butyl-hydroxyanisole and/or butyl-hydroxytoluene may be used as an antioxidant. The amount and type of antioxidant added might be in accordance with the particular requirements and may generally be in the range of from approximately 0.001 to 5% by weight, or from 0.01 to 4% by weight, or from 0.1 to 2% by weight, or from 0.1 to 1% by weight, based on the total weight of the composition.

Other representative amounts and types of excipients present in a pharmaceutical composition of the invention may be derived from the specific examples listed hereinafter.

Further enumerated Embodiments of the invention:

Embodiment 1 relates to a pharmaceutical composition suitable for topical administration, comprising:

(a) a TLR7 modulator comprising a benzo[f][1,7]naphthyridine derivative;

(b) a solvent,

(c) an emulsifier selected from lecithin (phosphatidyl choline), polyoxyl stearyl ether, caprylcaproyl polyoxyl glyceride, cetyl alcohol and cholesterol;

(d) a thickener or gelling agent selected from polyacrylates e.g. carbomer or carbopol, acryloyldimethyl taurate copolymer e.g. Sepineo@ P600, and xanthan gum;

(e) a buffer,

(f) an alkalizing agent,

(g) a preservative, and optionally

(h) an antioxidant.

Embodiment 2 relates to a composition according to embodiment 1, wherein the TLR7 modulator is a compound of formula (I):

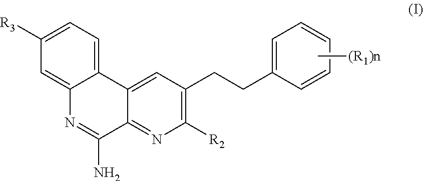

wherein each R₁, R₂, and R₃ are independently selected from H, —CH₃, —CH₂CH₃, —CF₃, —CH₂OH—OCH₃, —COOCH₃, —COOCH₂CH₃, F, Cl, Br, —CH₂OCH₃, —CH₂OCH₂CH₃, —N(CH₃)₂, —((O(CH₂)₂)₂—OH, —O(CH₂)₂—OH, —O(CH₂)₂—(PO₃H₂), —O(CH₂)₂—COOH, —O(CH₂)₂—CH(CH₃)₂, C₂-C₆-alkyl substituted with 1-3 substituents selected from —OH, —CH₃, cyclo-propyl, —O(CH₂)₂—COOH, —O(CH₂)₂—(PO₃H₂), —COOH, —COOCH₃, and —COOCH₂CH₃; and n is 0, 1,2 or 3; or a pharmaceutically acceptable salt thereof.

Embodiment 3 relates to a composition according to any one of the preceding embodiments (embodiments 1-2), wherein the solvent is selected from water, ethanol, propylene glycol, diethylene glycol monoethyl ether and benzyl alcohol.

Embodiment 4 relates to a composition according to any one of the embodiments 1-3, wherein the buffer is sodium citrate.

Embodiment 5 relates to a composition according to any one or the embodiments 1-4, wherein preservative is phenoxyethanol.

Embodiment 6 relates to a composition according to any one of the embodiments 1-5, wherein the alkalizing agent is sodium hydroxide.

Embodiment 7 relates to a composition according to any one of the preceding embodiments 1-6, wherein the antioxidant is butyl-hydroxyanisole.

Embodiment 8 relates to a composition according to any one of the preceding embodiments 1-7, wherein the TLR7 modulator is a compound of formula (II), (III) or (IV) or a pharmaceutically acceptable salt thereof:

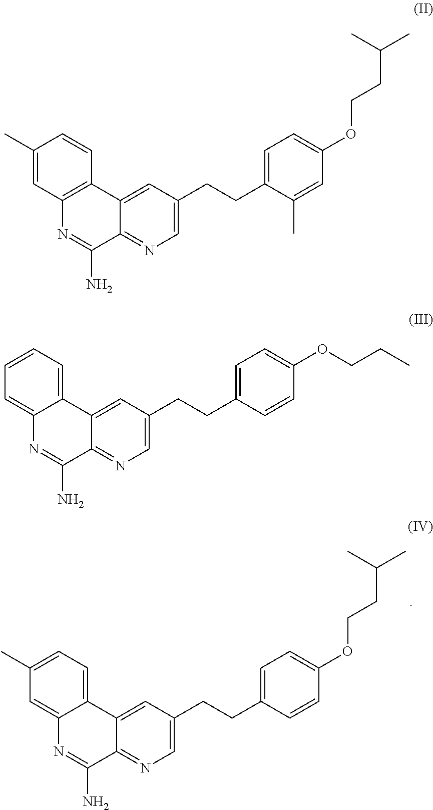

Embodiment 9 relates to a composition according to any one of the preceding embodiments 1-8 which is a cream or a gel.

Embodiment 10 relates to a composition according embodiment 1, comprising the following components:

| | | |
|---|---|---|
| Compound of formula (II) | 0.0075 | mg |
| Water purified | 19.235 | mg |
| Ethanol, anhydrous | 2.5 | mg |
| Diethylene glycol monoethyl ether | 1.0 | mg |
| Lecithin | 1.0 | mg |
| Sepineo ® P600 | 1.0 | mg |
| Phenoxyethanol | 0.125 | mg |
| Sodium citrate | 0.0725 | mg |
| Cholesterol | 0.03 | mg; and |
| Citric acid anhydrous | 0.03 | mg. |

Embodiment 11 relates to a composition according to embodiment 1, comprising the following components:

| | | |
|---|---|---|
| Compound of formula (II) | 0.0375 | mg |
| Water purified | 11.1625 | mg |
| Propylene glycol | 4.0 | mg |
| Diethylene glycol monoethyl ether | 2.5 | mg |
| Corn oil | 1.250 | mg |
| Benzyl alcohol | 0.750 | mg |
| Caprylcaproyl polyoxyl glycerides | 1.250 | mg |
| Polyoxyl stearyl ether | 1.75 | mg |
| (Brij ® S2 and Brij ® S721 in a ratio of 4:3) | | |
| Cetyl alcohol | 0.750 | mg |
| Phenoxyethanol | 0.250 | mg |
| Carbomer | 0.250 | mg |
| Xanthan gum | 0.05 | mg, and |
| NaOH (2N aqueous solution) | 1.0 | mg. |

Embodiment 12 relates to a composition according embodiment 1, consisting of the following components:

| | |
|---|---|
| Compound of formula (II) | 0.03% |
| Water purified | 76.94% |
| Ethanol, anhydrous | 10% |
| Diethylene glycol monoethyl ether | 4% |
| Lecithin | 4% |
| Sepineo ® P600 | 4% |
| Phenoxyethanol | 0.5% |
| Sodium citrate | 0.29% |
| Cholesterol | 0.12%; and |
| Citric acid anhydrous | 0.12%; |

wherein % refer to weight % of the total amount of the composition.

Embodiment 13 relates to a composition according to embodiment 1, consisting of the following components:

| Compound of formula (II) | 0.15% |
|---|---|
| Water purified | 44.65% |
| Propylene glycol | 16% |
| Diethylene glycol monoethyl ether | 10% |
| Corn oil | 5% |
| Benzyl alcohol | 3% |
| Caprylcaproyl polyoxyl glycerides | 5% |
| Polyoxyl stearyl ether | 7% |
| wherein said Polyoxyl stearyl ether consists of | |
| Brij ® S2 and Brij ® S721 in a ratio of 4:3 | |
| Cetyl alcohol | 3% |
| Phenoxyethanol | 1% |
| Carbomer | 1% |
| Xanthan gum | 0.2%, and |
| NaOH (2N aqueous solution) | 4%; |

wherein % refer to weight % of the total amount of the composition.

Embodiment 14 relates to a composition according to embodiment 1, consisting of the following components:

| | | |
|---|---|---|
| Compound of formula (II) | 0.0125 | mg |
| Water purified | 11.1625 | mg |
| Propylene glycol | 4.0 | mg |
| Diethylene glycol monoethyl ether | 2.5 | mg |
| Corn oil | 1.250 | mg |
| Benzyl alcohol | 0.750 | mg |
| Caprylcaproyl polyoxyl glycerides | 1.250 | mg |
| Polyoxyl stearyl ether | 1.750 | mg |
| Sodium hydroxide | 1.0 | mg |
| Cetyl alcohol | 0.750 | mg |
| Phenoxyethanol | 0.250 | mg |
| Carbomer | 0.250 | mg |
| Optionally Butylhydroxyanisole | 0.0250 | mg; and |
| Xanthan gum | 0.05 | mg. |

### Example 1

For the manufacture of a topical cream, the following amounts (in grams) were present in a batch consisting of 25 g (total amount). 1 gram cream hence comprises 0.3 mg of compound of formula (II). For a cream, wherein the concentration of compound of formula (II) contains 1 mg active ingredient per 1 gram of cream, compound No. (II) is present in an amount of 0.025 g per 25 g total amount of cream.

| | |
|---|---|
| Compound formula (II) | 0.0075 |
| | (corresponds to 0.3 mg/g cream) |
| Water purified | 19.235 |
| Ethanol, anhydrous | 2.5 |
| Diethylene glycol monoethyl ether | 1.0 |
| Lecithin | 1.0 |
| Sepineo ® P600 | 1.0 |
| Phenoxyethanol | 0.125 |
| Sodium citrate | 0.0725 |
| Cholesterol | 0.03 |
| Citric acid anhydrous | 0.03 |

Description of manufacturing (process was scaled up to about 25 kg batch size):

1. Citric acid anhydrous and sodium citrate were dissolved in purified water (=aqueous phase).

2. Compound of formula (II) was suspended in ethanol, (=lipophilic phase part 2) in a vessel.

3. Diethylene glycol monoethyl ether, lecithin, cholesterol and phenoxyethanol were mixed by stirring under heat (=lipophilic phase−part 1).

4. Thereupon the lipophilic phase—part 2 was transferred into the lipophilic phase—part 1 under stirring and heating. The vessel used in step 2 was flushed with remaining quantity of anhydrous ethanol.

5. The material from step 4 was then added to the material of step 1 i.e. aqueous phase under stirring and homogenization while cooling down.

6. Sepineo® P600 was added to the material of step 5 under stirring and homogenization. Stirring was continued until a homogenous cream was obtained.

7. The resulting cream was then transferred into a suitable vessel.

8. The cream was then filled into aluminium tubes (e.g. tubes comprising 10 g cream) and the tubes were closed.

Stability Assessment

Stability of the cream formulation described in example 1 was tested over a period of 12 months at a temperature of 25° C. Under these conditions no unacceptable amounts of degradation product(s) were identified.

### Example 2

For the manufacture of a topical cream, the following amounts (in gram) were present in a batch consisting of 25 g (total amount). 1 gram cream hence comprises 0.5 mg of compound No (II).

| | |
|---|---|
| Compound formula (II) | 0.0125 |
| | (corresponds to 0.5 mg/g cream) |
| Water purified | 11.1625 |
| Propylene glycol (PG) | 4.0 |
| Diethylene glycol monoethyl ether | 2.5 |
| (=Transcutol ® HP) | |
| Corn oil | 1.250 |
| Benzyl alcohol | 0.750 |
| Caprylcaproyl polyoxyl glycerides | 1.250 |
| (Labrasol ®) | |
| Polyoxyl stearyl ether | 1.750 |
| (Brij ® S2 and S721 ratio 4:3) | |
| Sodium hydroxide | 1.0 |
| Cetyl alcohol | 0.750 |
| Phenoxyethanol | 0.250 |
| Carbomer | 0.250 |
| Optionally Butylhydroxyanisole | 0.0250 |
| Xanthan gum | 0.05 |

Description of manufacturing (process was scaled up to about 25 kg batch size):

1. Xanthan gum was dispersed in propylene glycol to form xanthan gum premix. This xanthan gum premix was then transferred into water and the carbomer was added to form the aqueous phase.

2. The above mixture was stirred under heating until a homogenous gel was formed.

3. Compound of formula (II) was suspended in diethylene glycol monoethyl ether.

4. The lipophilic phase was then formed by mixing the following components: polyoxyl stearyl ether, corn oil, cetyl alcohol, capryl caproyl polyoxyl glyceride, benzyl alcohol, phenoxyethanol, butylated hydroxyl anisole and Compound of formula (II) suspension from step 3. The vessel used in step 3 was flushed with remaining diethylene glycol monoethyl ether and was added to said lipophilic phase.

5. The mixture of step 4 was then stirred until clear solution was formed.

6. The solution of step 5 was then added to the gel obtained in step 2, whereby the mixture was initially only stirred and thereafter homogenized under stirring.

7. 2 N NaOH solution was then added to the material of step 6, whereby stirring and homogenization was continued.

8. The bulk material so obtained in step 7 was then transferred into suitable vessel.

9. The cream obtained in step 7 was then filled into aluminium tubes which were sealed thereupon.

### Example 3

Similarly to example 2 the following composition was prepared consisting of:

| | | |
|---|---|---|
| Compound of formula (II) | 0.0375 | mg |
| Water purified | 11.1625 | mg |
| Propylene glycol | 4.0 | mg |
| Diethylene glycol monoethyl ether | 2.5 | mg |
| Corn oil | 1.250 | mg |
| Benzyl alcohol | 0.750 | mg |
| Caprylcaproyl polyoxyl glycerides | 1.250 | mg |
| Polyoxyl stearyl ether | 1.75 | mg |
| (Brij ® S2 and Brij ® S721 in a ratio of 4:3) | | |
| Cetyl alcohol | 0.750 | mg |
| Phenoxyethanol | 0.250 | mg |
| Carbomer | 0.250 | mg |
| Xanthan gum | 0.05 | mg, and |
| NaOH (2N aqueous solution) | 1.0 | mg. |

Stability Assessment

Stability of the cream formulation described in example 2 was tested over a period of 12 months at a temperature of 25° C. Under these conditions no unacceptable amounts of degradation product(s) were identified.

Tolerability Assessment

The galenic formulations of examples No. 1 and No. 2 (in each the concentration of the active ingredient was 0.25% and some ingredients were slightly adjusted) were tested topically on the skin of domestic pigs and the mean inflammatory scores were recorded over a period of 16 days. These were determined by assessing the sum of inflammation scores (changes in histopathology). As a comparison, commercially available Aldara® cream (5%) was taken (the compound of formula (II) is about 100 times more potent TLR7 agonist than Aldara®), and a standard galenic formulation (hereinafter “Standard Gel”) with the following composition details:

Standard Gel:

| | |
|---|---|
| Compound of formula (II) | 0.5% |
| Polyethyleneglycol 400 | 28.75% |
| Propylene glycol | 30% |
| Sepineo ® P 600 | 5% |
| Transcutol ® HP | 10% |
| Benzyl alcolhol | 3% |
| Phenoxyethanol | 1% |
| Oleyl alcohol | 1% |
| Dimethyl isosorbide | 10% |
| Labrasol ® | 10.5% |
| BHT | 0.1% |
| BHA | 0.1% |
| Tocopherol | 0.05% |
| Total amount | 100% (by weight) |

The following overall tolerability ranking was assessed:

| | Example 1 | Example 2 | Standard Gel | Aldara ® |
|---|---|---|---|---|
| Overall Tolerability | 3.7 | 2.7 | 11.7 | 13.3 |
| Ranking based upon | | | | |
| summed up | | | | |
| epidermal/dermal | | | | |
| inflammatory | | | | |
| histological changes. | | | | |
| The Total Scores are | | | | |
| indicated. | | | | |

The formulations or examples 1 and 2 were very well tolerated on domestic pig skin, whereas the Standard Gel and Aldara® showed distinct skin irritations as shown by the above indicated numerical scores.

In analogy to Example 1, further stable and well tolerated creams were prepared within the scope of the present invention in accordance to the below (% is weight % and refers to the total weight of a composition):

Ingredient

| | | | | | | |
|---|---|---|---|---|---|---|
| Compound of formula II | 0.1% | 0.06% | 0.04% | 0.1% | 0.1% | 0.07% |
| Lecithin | 4.0% | 4.0% | 4.0% | 4.0% | 8.0% | 8.0% |
| Cholesterol | 0.12% | 0.12% | 0.12% | 0.12% | 0.24% | 0.24% |
| Water purified (added up | Ad. 100% | Ad. 100% | Ad. 100% | Ad. 100% | Ad. 100% | Ad. 100% |
| to a total of 100%) | | | | | | |
| Ethanol | 10% | 10% | 10% | 10% | 10% | 10% |
| Phenoxyethanol | 0.5% | 0.5% | 0.5% | 0.5% | 0.5% | 0.5% |
| Diethylene glycol | 4% | 4% | 4% | 4% | 4% | 4% |
| monoethyl ether | | | | | | |
| Citric acid (anhydrous) | 0.12% | 0.12% | 0.12% | 0.12% | 0.12% | 0.12% |
| Trisodium citrate | 0.29% | 0.29% | 0.29% | 0.29% | 0.29% | 0.29% |
| Sepineo P600 | 4% | 4% | 4% | 5% | 4% | 4% |

1. A pharmaceutical composition suitable for topical administration, comprising:
(a) a TLR7 modulator comprising a benzo[f][1,7]naphthyridine derivative;
(b) a solvent,
(c) an emulsifier selected from lecithin (phosphatidyl choline), polyoxyl stearyl ether, caprylcaproyl polyoxyl glyceride, cetyl alcohol, and cholesterol;
(d) a thickener or gelling agent selected from polyacrylates e.g. carbomer or carbopol, acryloyldimethyl taurate copolymer e.g. Sepineo® P600, and xanthan gum;
(e) a buffer,
(f) an alkalizing agent,
(g) a preservative, and optionally
(h) an antioxidant.

2. The composition according to claim 1, wherein the TLR7 modulator is a compound of formula (I): 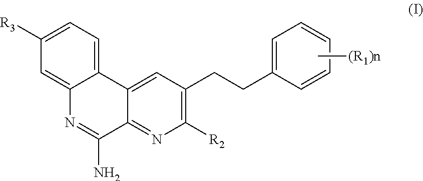
wherein
each R₁, R₂, and R₃ are independently selected from H, —CH₃, —CH₂CH₃, —CF₃, —CH₂OH—OCH₃, —COOCH₃, —COOCH₂CH₃, F, Cl, Br, —CH₂OCH₃, —CH₂OCH₂CH₃, —N(CH₃)₂, —((O(CH₂)₂)₂—OH, —O(CH₂)₂—OH, —O(CH₂)₂—(PO₃H₂), —O(CH₂)₂—COOH, —O(CH₂)₂—CH(CH₃)₂, C₂-C₆-alkyl substituted with 1-3 substituents selected from —OH, —CH₃, cyclo-propyl, —O(CH₂)₂—COOH, —O(CH₂)₂—(PO₃H₂), —COOH, —COOCH₃, and —COOCH₂CH₃; and
n is 0, 1, 2 or 3; or a pharmaceutically acceptable salt thereof.

3. The composition according to claim 1, wherein the solvent is selected from water, ethanol, propylene glycol, diethylene glycol monoethyl ether and benzyl alcohol.

4. The composition according claim 1, wherein the buffer is sodium citrate.

5. The composition according to claim 1, wherein preservative is phenoxyethanol.

6. The composition according to claim 1, wherein the alkalizing agent is sodium hydroxide.

7. The composition according to claim 1, wherein the antioxidant is butyl-hydroxyanisole.

8. The composition according to claim 1, wherein the TLR7 modulator is a compound of formula (II), (III) or (IV) or a pharmaceutically acceptable salt thereof:

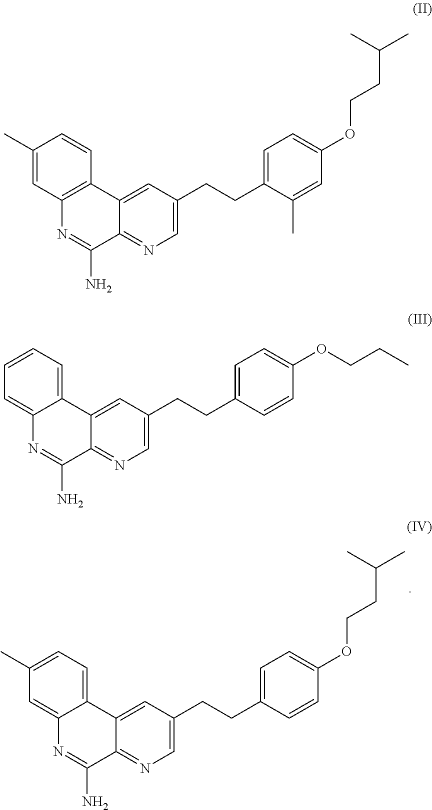

9. The composition according to claim 1 which is a cream or a gel.

10. The composition according claim 8, comprising the following components:

| | | |
|---|---|---|
| Compound of formula (II) | 0.0075 | mg |
| Water purified | 19.235 | mg |
| Ethanol, anhydrous | 2.5 | mg |
| Diethylene glycol monoethyl ether | 1.0 | mg |
| Lecithin | 1.0 | mg |
| Sepineo ® P600 | 1.0 | mg |
| Phenoxyethanol | 0.125 | mg |
| Sodium citrate | 0.0725 | mg |
| Cholesterol | 0.03 | mg; and |
| Citric acid anhydrous | 0.03 | mg. |

11. The composition according to claim 8, comprising the following components:

| | | |
|---|---|---|
| Compound of formula (II) | 0.0125 | mg |
| Water purified | 11.1625 | mg |
| Propylene glycol | 4.0 | mg |
| Diethylene glycol monoethyl ether | 2.5 | mg |
| Corn oil | 1.250 | mg |
| Benzyl alcohol | 0.750 | mg |
| Caprylcaproyl polyoxyl glycerides | 1.250 | mg |
| Polyoxyl stearyl ether | 1.750 | mg |
| Sodium hydroxide | 1.0 | mg |
| Cetyl alcohol | 0.750 | mg |
| Phenoxyethanol | 0.250 | mg |
| Carbomer | 0.250 | mg |
| Optionally Butylhydroxyanisole | 0.0250 | mg; and |
| Xanthan gum | 0.05 | mg. |

12. The composition according to claim 10, wherein the ratios of the components are maintained and the total amount of a composition is any fraction or any multiple thereof.

13. The composition of claim 8, which comprises in weight percent based upon the total weight of a composition:

| | |
|---|---|
| Compound of formula (II) | 0.1% |
| Water purified | added up to 100% |
| Ethanol, anhydrous | 10% |
| Diethylene glycol monoethyl ether | 4% |
| Lecithin | 4% |
| Sepineo ® P600 | 4% |
| Phenoxyethanol | 0.5% |
| Trisodium citrate | 0.29% |
| Citric acid (anhydrous) | 0.12%, and |
| Cholesterol | 0.12%. |

14. The composition of claim 8, which comprises in weight percent based upon the total weight of a composition:

| | |
|---|---|
| Compound of formula (II) | 0.1% |
| Water purified | added up to 100% |
| Ethanol, anhydrous | 10% |
| Diethylene glycol monoethyl ether | 4% |
| Lecithin | 4% |
| Sepineo ® P600 | 5% |
| Phenoxyethanol | 0.5% |
| Trisodium citrate | 0.29% |
| Citric acid (anhydrous) | 0.12%, and |
| Cholesterol | 0.12%. |

15. The composition of claim 8, which comprises in weight percent based upon the total weight of a composition:

| | |
|---|---|
| Compound of formula (II) | 0.1% |
| Water purified | added up to 100% |
| Ethanol, anhydrous | 10% |
| Diethylene glycol monoethyl ether | 4% |
| Lecithin | 8% |
| Sepineo ® P600 | 4% |
| Phenoxyethanol | 0.5% |
| Trisodium citrate | 0.29% |
| Citric acid (anhydrous) | 0.12%, and |
| Cholesterol | 0.24%. |

16. The composition according to claim 11, wherein the ratios of the components are maintained and the total amount of a composition is any fraction or any multiple thereof.
